(19) United States
(12) Patent Application Publication
(10) Pub. No.: US 2024/0082452 A1
(43) Pub. Date: Mar. 14, 2024

(54) DEVICE FOR DIFFUSING VOLATILE SUBSTANCES

(71) Applicant: ZOBELE HOLDING SPA, Trento (IT)

(72) Inventors: Marina Trias Lafuente, Barcelona (ES); Roberto Camarero Diez, Barcelona (ES); Joaquim Llorente Alonso, Barcelona (ES)

(73) Assignee: ZOBELE HOLDING SPA, Trento (IT)

(21) Appl. No.: 17/767,817

(22) PCT Filed: Oct. 8, 2020
(86) PCT No.: PCT/EP2020/078265

§ 371 Date: Apr. 8, 2022

(30) Foreign Application Priority Data
Oct. 9, 2019 (ES) P201930881

(51) Int. Cl. A61L 9/12

(52) U.S. Cl. CPC A61L 9/127; A61L 2209/133

(57) ABSTRACT

The device for diffusing volatile substances comprises a container (1) containing a liquid with said volatile substances, a base element (2) that closes said container (1), and a wick assembly (3) that is impregnated with said volatile substances, wherein said wick assembly (3) is removably fixed to said base element (2) and comprises a projection (31) that activates the opening of a valve (11) of said container (1). It allows providing a volatile substances device that prevents leaks and ensures correct dosage and evaporation of volatile substances.

The present invention relates to a device for the diffusion of volatile substances.

### BACKGROUND OF THE INVENTION

There are devices on the market for diffusing volatile substances, such as static air fresheners with inverted positioning. As they are products with inverted positioning, the difficulty of the solutions consists in the correct dosage of the volatile substances.

In existing products, this solution usually consists of an intermediate wick fixed to the container by means of a plug.

By placing the bottle on the base, this intermediate wick comes into contact with a secondary wick. The intermediate wick functions as a dispenser, and the secondary wick functions as the evaporation surface.

A drawback of these devices is that the intermediate wick forms part of the fragrance container, so that good contact between the intermediate wick and the secondary wick cannot be ensured.

In addition, another drawback is that dripping may occur at the time of activation, and, in addition, the product is usually single-use.

Therefore, an object of the present invention is to provide a device for volatile substances that prevents leaks and that ensures correct dosing and evaporation of volatile substances.

### DESCRIPTION OF THE INVENTION

With the device for diffusing volatile substances according to the invention, the aforementioned drawbacks are solved, presenting other advantages that will be described below.

The device for diffusing volatile substances according to the present invention comprises:

The device for diffusing volatile substances according to the present invention comprises:
a container containing a liquid with such volatile substances,
a base element that closes said container, and
a wick assembly that is impregnated with said volatile substances,
   wherein said wick assembly is removably fixed to said base element and comprises a projection that activates the opening of a valve of said container.

According to a preferred embodiment, said wick assembly defines a base from which said protrusion projects.

Furthermore, according to said embodiment, said wick assembly comprises lateral extensions.

Said wick assembly advantageously comprises a fixing piece, which connects the projection to the base and removably fixes the wick assembly to the base element.

To facilitate the evaporation of volatile substances, said base element comprises a plurality of evaporation holes, and said evaporation holes are preferably arranged on the side wall of said base element.

According to a preferred embodiment, said base element comprises a housing in which said fixing piece is removably fixed.

The device for diffusing volatile substances according to the present invention has at least the following advantages:
It avoids leaks,
It ensures a correct dosage and evaporation of volatile substances,
The wick incorporates fixing and easy release means without the need for the user to come into contact with the impregnated wick,
It facilitates the reuse of parts.

### BRIEF DESCRIPTION OF THE DRAWINGS

For a better understanding what has been stated, some drawings are attached in which, schematically and only as a non-limiting example, a practical case of embodiment is represented.

FIG. 1 is an exploded perspective view of the device for diffusing volatile substances according to the present invention; and

FIG. 2 is an exploded longitudinal sectional view of the device for diffusing volatile substances according to the present invention.

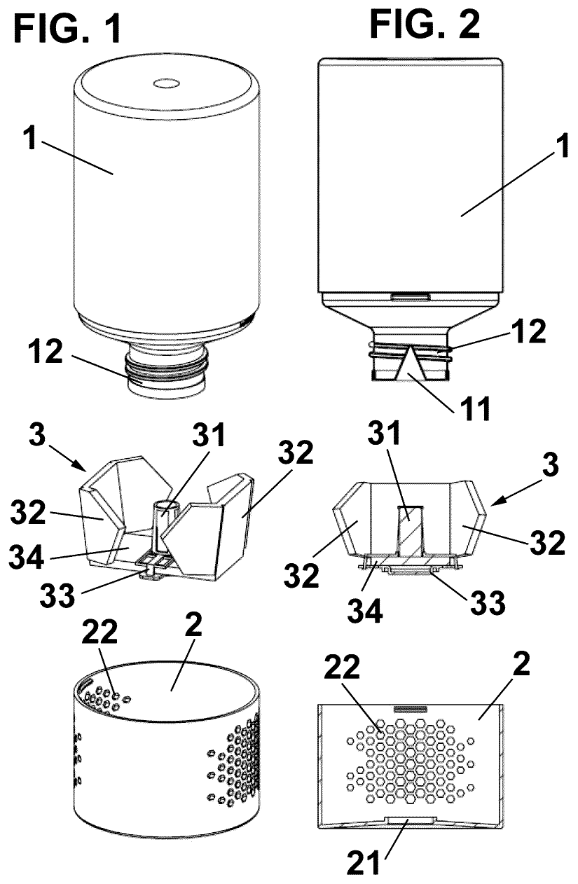

### DESCRIPTION OF A PREFERRED EMBODIMENT

As shown in the figures, the device for diffusing volatile substances comprises a container 1 containing a liquid with the volatile substances.

Said container 1 comprises a valve 11, which is actuated before the use of the diffusion device, as will be explained later, and a mouth 12 provided with an external thread.

The device for diffusing volatile substances also comprises a base element 2. This base element 2 is used to place the device in an inverted position, that is, with the base element 2 in the lower position, as shown in the figures.

To facilitate evaporation, the base element 2, which is preferably cylindrical in shape, comprises a plurality of evaporation holes 22, which are arranged in the side wall of the base element 2, in accordance with the embodiment shown.

The device for diffusing volatile substances also comprises a wick assembly 3, which comprises a projection 31 that activates the valve 11 of container 1, opening the outlet of the liquid from said container through the valve 11.

In this way, according to the embodiment shown, the liquid with the volatile substances is impregnated in the wick 3 through this projection 31.

Said wick assembly 3 defines a base 34 and lateral extensions 32, as can be seen in the figures.

In addition, the projection 31 is connected to the base 34 by means of a fixing piece 33, which allows the fixing in a removable manner to said base element 2, which comprises a housing 21, which houses the lower part of the fixing piece 33.

Said fixing piece 33, as shown in the figures, is preferably a clamp or the like that surrounds the projection 31 and the base 34.

When it is desired to use the device for diffusing volatile substances according to the present invention, first the wick assembly 3 is placed in its position inside the base element 2, fixed in a removable manner, that is to say, it can be removed when desired to be replaced with another wick assembly.

Said wick assembly 3 shall be replaced as many times as necessary, as it is a spare element.

Next, the base element 2 will be coupled to a container 1. In doing so, the projection 31 of the wick assembly 3 shall be inserted into the valve 11, opening it.

By placing the diffusion device in its position of use, that is, in the position represented in the figures with the base element 2 in its lower part, the liquid shall pass through the valve 11 and impregnate the wick assembly 3 with said liquid and the volatile substances shall diffuse or evaporate to the environment.

Said diffusion or evaporation is performed through the evaporation holes 25, in the embodiment shown, although it could also be carried out through a gap between the base element 2 and the container 1.

Despite the fact that reference has been made to a specific embodiment of the invention, it is obvious to a person skilled in the art that the device described for diffusing volatile substances is susceptible to numerous variations and modifications, and that all the mentioned details can be substituted by others technically equivalent, without departing from the scope of protection defined by the appended claims.

1. A device for diffusing volatile substances, comprising:
a container containing a liquid with said volatile substances,
a base element that closes said container, and
a wick assembly that is impregnated with said volatile substances,
characterized in that said wick assembly is fixed in a removable manner to said base element and comprises a projection that activates the opening of a valve of said container.

2. The device for diffusing volatile substances according to claim 1, wherein said wick assembly defines a base from which said projection projects.

3. The device for diffusing volatile substances according to claim 1, wherein said wick assembly comprises lateral extensions.

4. The device for diffusing volatile substances according to claim 2, wherein said wick assembly comprises a fixing piece, which joins the projection to the base and removably fixes the wick assembly to the base element.

5. The device for diffusing volatile substances according to claim 1, wherein said base element comprises a plurality of evaporation holes.

6. The device for diffusing volatile substances according to claim 5, wherein said evaporation holes are arranged on the side wall of said base element.

7. The device for diffusing volatile substances according to claim 4, wherein said base element comprises a housing in which said fixing piece is removably fixed.

8. The device for diffusing volatile substances according to claim 2, wherein said wick assembly comprises lateral extensions.
